(19) United States
(12) Patent Application Publication
(10) Pub. No.: US 2013/0045500 A1
(43) Pub. Date: Feb. 21, 2013

(54) CELL EVALUATION METHOD

(71) Applicants: Satoru Tanaka, Kanagawa (JP); Ruriko Sakamoto, Kanagawa (JP); Tetsuya Nakatsura, Chiba (JP); Manami Shimomura, Chiba (JP)

(72) Inventors: Satoru Tanaka, Kanagawa (JP); Ruriko Sakamoto, Kanagawa (JP); Tetsuya Nakatsura, Chiba (JP); Manami Shimomura, Chiba (JP)

(73) Assignees: NATIONAL CANCER CENTER, Tokyo (JP); SCIVAX CORPORATION, Kanagawa (JP)

(21) Appl. No.: 13/212,817

(22) Filed: Aug. 18, 2011

(51) Int. Cl. C12Q 1/02

(52) U.S. Cl. 435/29

(57) ABSTRACT

The present invention provides a method of figuring out an effect of a medicine exerting on the same cell quantitatively over a time. A cell evaluation method includes a step of culturing cells on a cell-culture base material that is capable of culturing spheroids, and a step of measuring a change over a time in the number of the spheroids formed through the former step or a percentage of the spheroids formed through the former step relative to an entire cultured cell.

### BACKGROUND OF THE INVENTION

1. Field of the Invention

The present invention relates to a method for evaluating a survival rate, etc., of a cell by measuring the number of spheroids on a culture vessel.

2. Description of the Related Art

In recent years, an evaluation system using cultured cells has an extremely important role in an evaluation of medicine susceptibility and toxicity in the field of medicine discovery.

For example, the survival rate, proliferation potency, and migration capability of a cell are important factors in a screening of an anticancer agent.

Examples of the method for evaluating the survival rate of a cell are a method of staining a dead cell with a pigment like trypan blue, a method of measuring an enzyme activity derived from a living cell through a color reaction utilizing a reduction of tetrazolium salt, and a method of measuring an ATP amount in a cell with luciferase.

However, these methods perform physical and chemical processes on a cell when the color reaction such as staining, coloring, or light emission is caused. For this reason, it is difficult to keep culturing the cell after such processes. In other words, a state of the cell at the time of the measurement is merely evaluated in a pin-point manner, and it is difficult to figure out the effect of a medicine, etc., exerting on the same cell quantitatively over a time.

It is an object of the present invention to provide a cell evaluation method that is capable of evaluating the survival rate, etc., of a cell without giving any damage to the cell in a state in which continuation of the cell culturing is possible.

### SUMMARY OF THE INVENTION

Inventors of the present invention found out that the number of spheroids formed or the percentage of the spheroids formed relative to an entire cultured cell changes in accordance with a contact frequency of the cells caused by the migration capability when the cell is cultured on the cell-culture base material includes a predetermined concavo-convex structure that functions as a cell adhesive surface. Moreover, the inventors found out that there is an inverse correlation between the number or the percentage of the spheroids and the survival rate of the cultured cell, and completed the present invention that is a cell evaluation method using spheroids as a parameter.

A subject matter of the present invention is as follows.

The cell evaluation method of the present invention includes a step of culturing cells on a cell-culture base material that is capable of culturing spheroids, and a step of measuring a change over a time in the number of the spheroids formed through the former step or a percentage of the spheroids formed through the former step relative to an entire cultured cell.

In this case, the cell-culture base material that includes a predetermined concavo-convex structure that functions as a cell adhesive surface may be used.

It is preferable that the concavo-convex structure should include a plurality of unit structures arranged regularly, the unit structure being in a predetermined planar shape.

Moreover, it is preferable that the concavo-convex structure should include a plurality of unit structures arranged regularly, the unit structure having a width between adjoining unit structures that is equal to or smaller than 3 μm, being in a polygonal planer shape, and having a minimum internal diameter that is equal to or smaller than 3 μm.

Furthermore, it is preferable that the cell evaluation method according to the present invention should further include a step of picking up an image of the cultured cell with a predetermined visual field.

According to the present invention, it is possible to quantitatively evaluate a change in the survival rate of cells over a time without giving any damage to the cells.

### BRIEF DESCRIPTION OF THE DRAWINGS

FIG. 1 is a plan view showing a concavo-convex structure of a culture base material used in the present invention;

FIG. 2 is a microscopic photograph of an effect of trastuzumab exerting on a breast-cancer-derived cell line BT474 observed over a time;

FIG. 3 is a graph showing an effect of trastuzumab exerting on a breast-cancer-derived cell line BT474 indicated as an ATP amount in a cell;

FIG. 4 is a graph showing an effect of trastuzumab exerting on a breast-cancer-derived cell line BT474 indicated as the number of spheroids;

FIG. 5 is a microscopic photograph of an effect of paclitaxel exerting on a breast-cancer-derived cell line BT474 observed over a time;

FIG. 6 is a graph showing an effect of paclitaxel exerting on a breast-cancer-derived cell line BT474 indicated as an ATP amount in a cell;

FIG. 7 is a graph showing an effect of paclitaxel exerting on a breast-cancer-derived cell line BT474 indicated as the number of spheroids;

FIG. 8 is a graph showing effects of trastuzumab and paclitaxel exerting on a breast-cancer-derived cell line T47D indicated as an ATP amount in a cell; and

FIG. 9 is a graph showing effects of trastuzumab and paclitaxel exerting on a breast-cancer-derived cell line T47D indicated as the number of spheroids.

### DETAILED DESCRIPTION OF THE PREFERRED EMBODIMENT

A detailed explanation will be given of the present invention in the following preferred embodiment.

A cell evaluation method of the present invention includes a step of culturing cells on a cell-culture base material that is capable of culturing spheroids, and a step of measuring a change over a time in the number of the spheroids formed through the above-described step or a percentage of the spheroids formed through the above-described step relative to an entire cultured cell. The spheroid in the present invention means an aggregation of cells which are gathered and aggregated one another three-dimensionally.

The cell-culture base material that is capable of culturing the spheroids is not limited to any particular kind as long as it can suppress an adhesiveness with a cell in comparison with a culture base material that is used for a normal monolayer culturing, and for example, one having modified hydrophilic or hydrophobic property of a surface thereof can be used.

Moreover, the cell-culture base material can have a predetermined concavo-convex structure that functions as a cell adhesive surface.

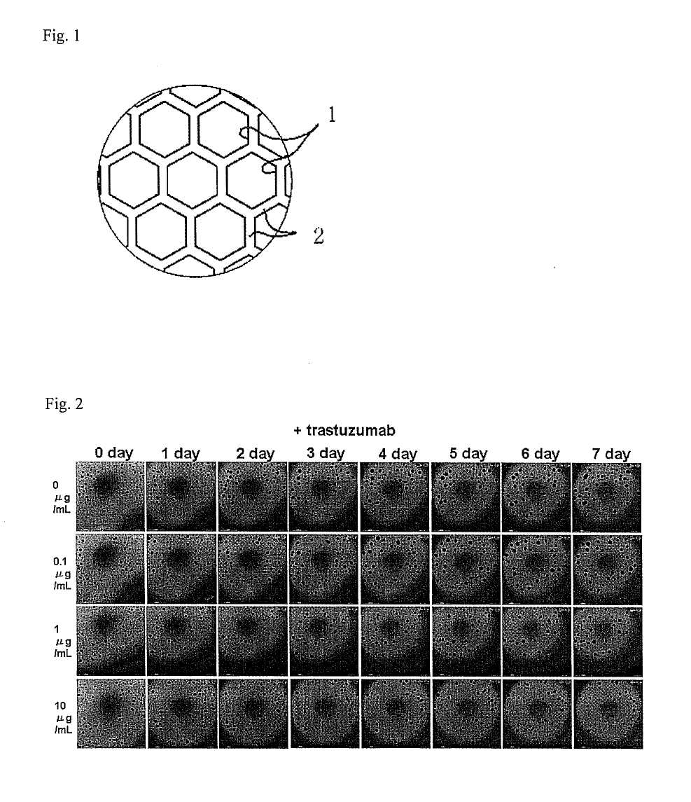

The concavo-convex structure can be in various kinds of shapes, such as a linear shape (line-and-space), a pillar shape, and a hall shape, in accordance with a characteristic of the cultured cell, but preferably, a structure that has a plurality of unit structures 1 arranged regularly each of which is a predetermined planar shape is preferable. For example, as shown in FIG. 1, a plurality of unit structures 1 with a polygonal planer shape can be arranged successively. At this time, a regular triangle, a square, a regular polygonal shape like regular hexagon and a circle are more preferable since those can grow the cell on a structure that is isotropically uniform. It is possible to combine the concavo-convex structure like a pillar shape and a hall shape with the concavo-convex structure that is formed of the unit structures 1 in a planar shape. Note that from the standpoint of simulating the cultured cell to be in a state in a living body, a smaller width of a line 2 is preferable, such as equal to or smaller than 3 μm, equal to or smaller than 2 μm, equal to or smaller than 1 μm, equal to or smaller than 700 nm, equal to or smaller than 500 nm, and equal to or smaller than 250 nm. This is because it is expected that the smaller the width of the line 2 becomes, the more the cell that is adhered to the concavo-convex structure surface forms the spheroids while growing a large number of pseudopods.

Moreover, the depth of the unit structure 1 is formed in various sizes, such as equal to or larger than 1 nm, equal to or larger than 10 nm, equal to or larger than 100 nm, equal to or larger than 200 nm, equal to or larger than 500 nm, equal to or larger than 1 μm, equal to or larger than 10 μm, and equal to or larger than 100 μm, in accordance with a characteristic of a cell to be cultured. Furthermore, there are various kinds of an aspect ratio of the concavo-convex, such as equal to or more than 0.2, equal to or more than 0.5, equal to or more than 1, and equal to or more than 2.

It is preferable that the minimum internal diameter (preferably maximum internal diameter) of the unit structure 1 should be equal to or smaller than 3 μm, and the smaller diameter is preferable, such as equal to or smaller than 2 μm, equal to or smaller than 1 μm, equal to or smaller than 700 nm, equal to or smaller than 500 nm, and equal to or smaller than 250 nm. The internal diameter means a distance between two parallel lines that circumscribe the unit structure 1. Therefore, the minimum internal diameter means the shortest distance between two parallel lines that circumscribe the unit structure 1, and the maximum internal diameter means the longest distance between two parallel lines that circumscribe the unit structure 1. For example, when the unit structure 1 is a regular hexagon, a distance between two parallel and opposite sides becomes the minimum internal diameter, and a distance between opposing vertexes becomes the maximum internal diameter. Furthermore, when the unit structure 1 is a rectangle, a length of a short side becomes the minimum internal diameter, and a length of a diagonal line becomes the maximum internal diameter.

The concavo-convex structure can be formed by any technique, but for example, nano-imprinting, solution-casting, etching, blasting, and corona discharging can be applied. In this case, nano-imprinting is preferable since it can control a shape, etc., more precisely.

The material of the cell-culture base material is not limited to any particular one as long as it is nontoxic to the cell, and for example, “polystyrene”, “polyethylene”, “polypropylene”, “polyimide”, “biodegradable polymer, such as a polylactic acid, polylactic acid-polyglycolic acid copolymer, and polycaprolactone”, “cyclic-olefin-based thermoplastic resin, such as cyclic-olefin copolymer (COC) and cyclic-olefin polymer (COP)”, “an acrylate resin”, “other resins, such as a photo-curable resin and a thermo-curable resin”, a “metal like aluminium oxide”, a “glass”, a “silica glass”, and a “silicon” can be used. Moreover, ones having a covering layer of a “resin”, a “photoresist”, or a “metal like aluminium oxide” formed on a surface of a base material main body made of silicon, or glass can also be used.

Moreover, the surface of the cell-culture base material may be processed to control the adhesiveness of the cell like ultraviolet irradiation, gamma-ray irradiation, plasma irradiation, and coating of various kinds of materials, etc., as long as such a surface can keep serving as the cell adhesive surface.

The cell to be used in the present invention is not limited to any particular one, but for example, an adipose cell, a hepatic cell, a renal cell, a pancreatic cell, a mammary cell, an endothelial cell, an epithelial cell, a smooth muscle cell, a myoblast cell, a cardiac muscle cell, a nerve cell, a glia cell, a dendritic cell, a cartilage cell, an osteoblastic cell, an osteoclastic cell, a bone cell, a fibroblastic cell, and a variety of hematopoietic cells that include a variety of precursor cells and stem cells, and others like a mesenchymal progenitor cell, a stem cell, an ES cell, and a variety of cancer cells, etc., can be used.

Culturing of a cell can be performed through the same culturing procedures as that of an operation normally performed.

The number of spheroids can be measured through an observation of the spheroids on the cell-culture base material using a biological microscope like a phase-contrast microscope. Also, the percentage of the spheroids relative to the entire cultured cell can be measured through a calculation of the number of spheroids or the number of cells that have not formed spheroids relative to the total number that is a sum of the number of spheroids and the number of cells which have not formed spheroids. Note that these measurements can be performed on cells that exist in a predetermined area under a microscope.

The reason why the survival rate, etc., of the cell can be evaluated through the measurement of the number of the spheroids cultured on the cell-culture base material having a predetermined concavo-convex structure is based on the following reasons.

First, it is expected that the cultured cell can elongate a large number of pseudopods on the cell-culture base material having the predetermined concavo-convex structure, so that the migration capability of the cell increases and the cells are likely to gather one another, and thus the spheroids are likely to be formed. Moreover, since the formed spheroids also migrate, the spheroids are also fused one another. Therefore, the higher the migration capability of the cultured cell is, the larger the size of the spheroid becomes, and the less the total number of the spheroids per unit area becomes.

On the other hand, it is expected that such spheroid formation needs an ATP. Hence, the inventors of the present invention thought that a change in the number of the spheroids per unit area and a change in the survival rate of the cultured cell are in a correspondence relation, and thus evaluation of the cell like an anticancer agent susceptibility is possible by measuring the number of the spheroids.

Since, the method of the present invention can evaluate the cell only through the measurement of the number of the spheroids without performing physical and chemical processes to the cell which are carried out in the case of the related art, it brings about an effect that the state of the cell can be checked quantitatively over a time. That is, the method of the present invention enables precise analysis in detail on not only the final effect of a medicine but also a mechanism until an effect of medicine becomes apparent and a differentiation tendency of the cell after a medicine is administrated, and can be a remarkably useful tool in the tailor-made medicine therapy.

The number of the spheroids can be visually performed, but can be performed only by picking up of an image of the cells with a predetermined visual field. By picking up an image thereof, a dead time between measurements can be eliminated, and it is possible to obtain a highly reliable data. Moreover, it is preferable to use a microscope or a camera for image pickup which are capable of both culturing and image pickup at the same time in a CO₂ incubator. Furthermore, it is useful for a high-throughput assay if a system that has programmed functions of an image processing and a data processing and analysis.

Note that by measuring the total of the number of the spheroids and the number of the cells that have not formed the spheroids, and by checking the change in the total number, evaluation of the cell like the survival rate may be performed.

Next, the present invention will be explained in detail with specific examples. However, the present invention is not limited to the following explanation.

### First Example

A human breast-cancer-derived cell line BT474 was suspended in a culture medium (NCM-M made by SCIVAX Co., Ltd.), and seeded on a NanoCulture (registered mark) plate (made by SCIVAX Co., Ltd., 96-well plate, material of concavo-convex structure surface: cyclic-olefin polymer (COP), planar shape of concavo-convex structure: regular hexagon, width between unit structures (line width): 700 nm, minimum internal diameter of unit structure: 3 μm, and depth: 1 μm) at 1×10⁴ cells/well, and incubated at 37° C. and 5% CO₂.

On the third day of the incubation, trastuzumab (made by CHUGAI PHARMACEUTICAL CO., LTD.) which was a kind of anticancer agent was added to each well so that the concentrations became 0 μg/mL, 0.1 μg/mL, 1.0 μg/mL, and 10 μg/mL, respectively, and then the incubation was continued for 7 days.

BT474 is a cell that overexpresses HER2 which is glycoprotein present on the cell surface, and trastuzumab is an anticancer agent that exerts an antitumor effect when specifically bound with HER2.

The effect of the medicine on the formation of spheroids in each concentration from the day zero to the seventh day after the medicine was added were observed through a time-lapse microscope, and the image pickup results are shown in FIG. 2. After the medicine was added, it becomes clear that the number of the spheroids changes depending on the medicine concentration as the time advances.

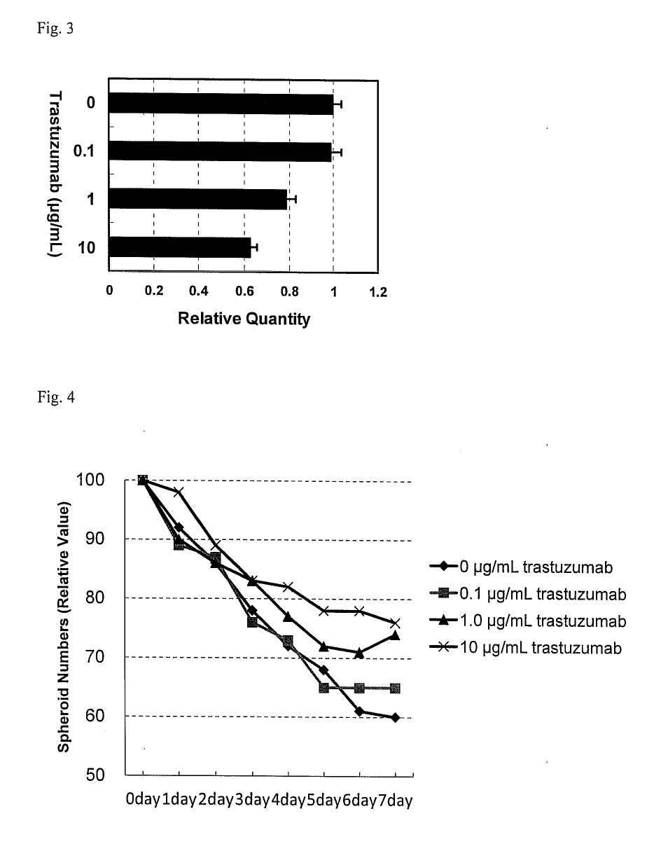

Moreover, the survival rate of the cell on the tenth day (the seventh day after medicine was added) was evaluated based on an assay of an ATP in the cell using CellTiter-Glo Luminescent Cell Viability Assay (Promega KK.) and calculated as a relative value, and the results are shown in FIG. 3.

Furthermore, the image of the change in the number of spheroids in each concentration from the day zero to the seventh day after the medicine was added was picked up using the time-lapse microscope, and the number of the spheroids was counted and taken as a relative value, and the results are shown in FIG. 4.

Based on these measurement results, it becomes clear that the value of survival rate of the cell indicated on the basis of the assay of an ATP and the number of the spheroids has a clear inverse correlation.

### Second Example

Like the first example, a human breast-cancer-derived cell line BT474 was incubated, and on the third day of the incubation, paclitaxel (made by Bristol-Myers Squibb Company) which was a kind of anticancer agent was added to each well so that the concentrations became 0 μg/mL, 0.06 μg/mL, 0.60 μg/mL, and 6.00 μg/mL, respectively, and then the incubation was continued for 7 days.

Paclitaxel is an anticancer agent that exerts an antitumor effect by inhibiting a cell division through an action on a microtubule.

The effect of the medicine on the formation of spheroids in each concentration from the day zero to the seventh day after the medicine was added was observed through a time-lapse microscope, and the image pickup results are shown in FIG. 5.

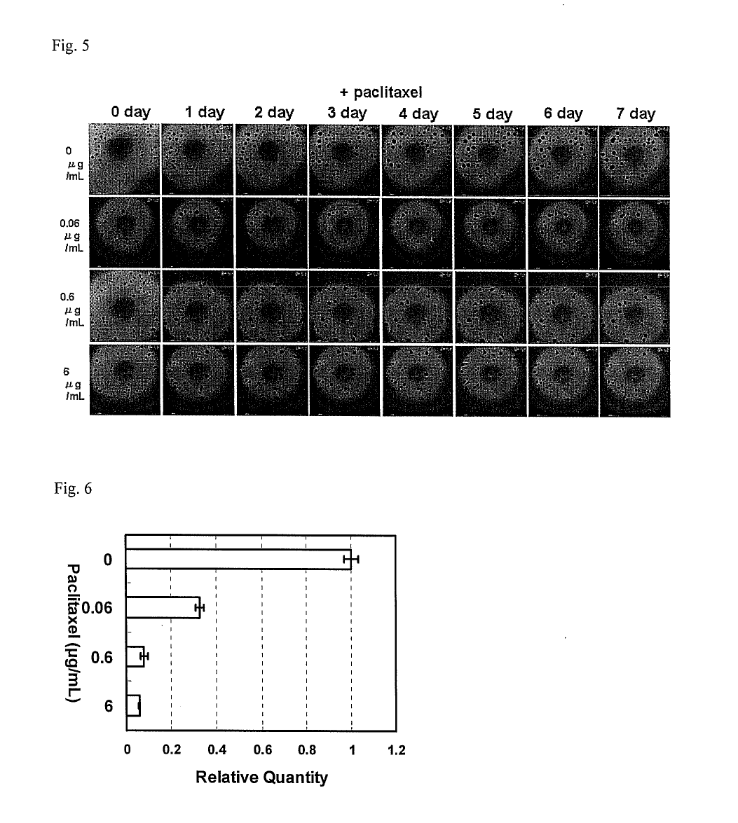

Moreover, the survival rate of the cell on the tenth day (the seventh day after medicine was added) was evaluated based on the assay of the ATP in the cell using CellTiter-Glo Luminescent Cell Viability Assay (Promega KK.) and calculated as a relative value, and the results are shown in FIG. 6.

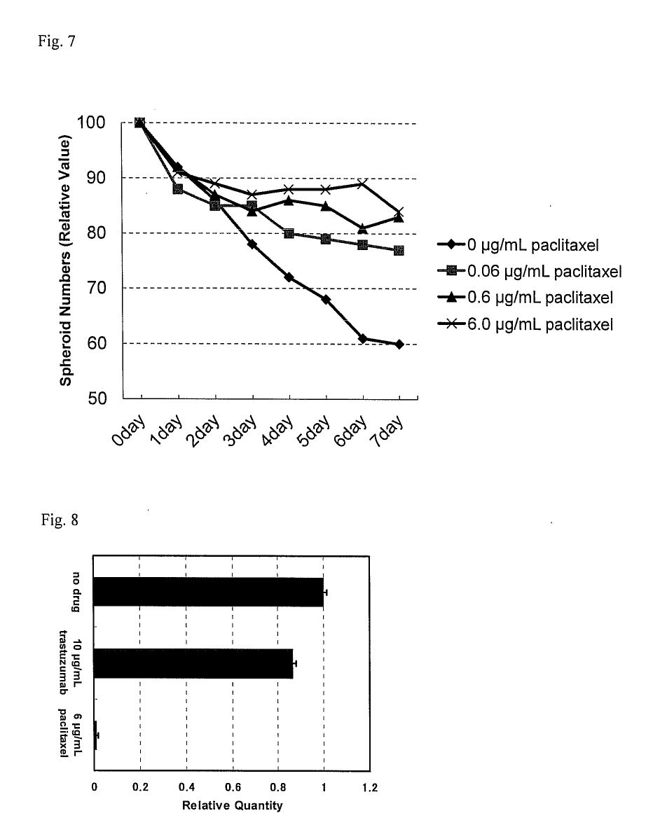

Furthermore, the image of the change in the number of spheroids in each concentration from the day zero to the seventh day after the medicine was added was picked up using the time-lapse microscope, and the number of the spheroids was counted and taken as a relative value, and the results are shown in FIG. 7.

Based on these measurement results, it becomes clear that the value of the survival rate of the cell indicated on the basis of the assay of an ATP and the number of the spheroids has the clear inverse correlation in paclitaxel like trastuzumab.

### Third Example

A human breast-cancer-derived cell line T47D was suspended in a culture medium (NCM-M made by SCIVAX Co., Ltd.), and seeded on a NanoCulture (registered mark) plate (made by SCIVAX Co., Ltd., 96-well plate, material of concavo-convex structure surface: cyclic-olefin polymer (COP), planar shape of concavo-convex structure: regular hexagon, width between unit structures (line width): 700 nm, minimum internal diameter of unit structure: 3 μm, and depth: 1 μm) at 1×10⁴ cells/well, and incubated at 37° C. and 5% CO₂.

T47D is a cell that expresses less HER2 glycoprotein compared to BT474 that is the same breast-cancer cell line.

On the third day of the incubation, trastuzumab (made by CHUGAI PHARMACEUTICAL CO., LTD.) was added so that the concentration thereof became 10 μg/mL and paclitaxel (made by Bristol-Myers Squibb Company) was added so that the concentration thereof became 6.0 μg/mL to each well, and then the incubation was continued for 7 days.

The survival rate of the cell on the tenth day (the seventh day after medicines were added) was evaluated based on the assay of the ATP in the cell using CellTiter-Glo Luminescent Cell Viability Assay (Promega KK.) and calculated as a relative value, and the results are shown in FIG. 8.

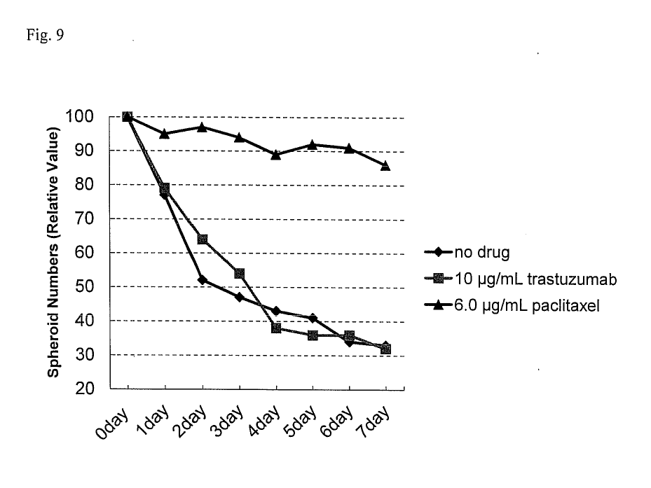

Moreover, the image of the change in the number of spheroids with each medicine from the day zero to the seventh day after the medicines were added was picked up using the time-lapse microscope, and the number of the spheroids was counted and taken as a relative value, and the results are shown in FIG. 9.

It is expected that paclitaxel (made by Bristol-Myers Squibb Company) that acts on a microtubule also shows an antitumor effect in the case of T47D like BT474. On the other hand, it is expected that, in the case of T47D which does not overexpress HER2, trastuzumab that has a specific action mechanism on HER2 does not show the anticancer effect unlike BT474.

Throughout the presence of the clear inverse correlation between the ATP amount in the cell and the number of spheroids, it was possible to confirm the expected effects that were shown in the above-description (FIG. 8 and FIG. 9). That is to say, it is understood that the method of the present invention can be an excellent tool for a screening of an anticancer agent.

1. A cell evaluation method comprising the steps of:
(a) culturing cells on a cell-culture base material that is capable of culturing spheroids;
(b) measuring a change over time in the number of the spheroids formed through step (a) or a percentage of the spheroids formed through step (a) relative to a starting cell population which was used to seed the cell-culture base material; and
(c) evaluating a cell survival rate or a drug susceptibility from data based on a measurement result of step (b).

2. The cell evaluation method according to claim 1, wherein the cell-culture base material includes a predetermined concavo-convex structure that functions as a cell adhesive surface.

3. The cell evaluation method according to claim 2, wherein the concavo-convex structure includes a plurality of unit structures arranged regularly, the unit structure being in a predetermined planar shape.

4. The cell evaluation method according to claim 2, wherein the concavo-convex structure includes a plurality of unit structures arranged regularly, the unit structure having a width between adjoining unit structures that is equal to or smaller than 3 μm, a polygonal planer shape, and having a minimum internal diameter that is equal to or smaller than 3 μm.

5. The cell evaluation method according to claim 1, further comprising a step of taking an image or photograph of a cell or a spheroid cultured on the cell-culture base material within a predetermined area or region.
